(19) United States
(12) Patent Application Publication
(10) Pub. No.: US 2022/0001146 A1
(43) Pub. Date: Jan. 6, 2022

(54) CATHETER NEEDLE ASSEMBLY WITH ENCLOSABLE NEEDLE

(71) Applicant: I-V Access Technology, Inc., San Francisco, CA (US)

(72) Inventors: Jacob HENTZLER, Morgan Hill, CA (US); James HALE, Los Osos, CA (US)

(73) Assignee: I-V Access Technology, Inc., San Francisco, CA (US)

(21) Appl. No.: 17/447,786

(22) Filed: Sep. 15, 2021

Related U.S. Application Data
(63) Continuation of application No. PCT/US2020/023700, filed on Mar. 19, 2020.
(60) Provisional application No. 62/820,716, filed on Mar. 19, 2019.

(51) Int. Cl. A61M 25/06; A61M 29/00; A61M 25/00

(52) U.S. Cl. CPC A61M 25/0606; A61M 25/0097; A61M 25/0693; A61M 29/00

(57) ABSTRACT

A needle catheter insertion device that includes a catheter-dilator assembly with a needle therethrough, where the dilator and needle are moveable relative to each other such that the needle catheter insertion device can be moved from a ready position, with the tip of the needle exposed, to a shielded position with the dilator encasing the needle. Positioning of the needle catheter insertion device into the shielded position can be accomplished by a manual actuation of the dilator into a locked configuration within a hub of the needle.

### CROSS-REFERENCE TO RELATED APPLICATIONS

This application is a continuation of U.S. Patent Application Number PCT/US2020/023700 filed Mar. 19, 2020, which claims priority to U.S. Provisional Patent Application No. 62/820,716 filed Mar. 19, 2019, the contents of which are incorporated herein by reference in their entireties.

### BACKGROUND OF THE INVENTION

Over-the-needle catheters generally include a cannula with a sharp tip that extends through a lumen of a catheter tubing, with the sharp tip projecting out of the distal end of the tubing. The medical provider advances the sharp tip of the cannula through the skin and into a blood vessel. The distal end of the catheter advances into the vessel upon further advancement of the cannula (also referred to as the needle). Once the catheter tubing is in the vessel, the caregiver withdraws the needle while leaving the catheter within the vessel and a hub of the catheter exposed for accessing the vessel and as a for connection to deliver or withdraw medical fluids from the vessel.

Caregivers face a significant risk of accidental needle sticks after removal of the needle cannula from the catheter. Clearly, such risks involve being exposed to any number of blood-borne diseases that could be transmitted by exposure to blood and other fluids of the patient. Such blood-borne diseases include, but are not limited, to human immunodeficiency virus (HIV), hepatitis B (HBV) and hepatitis C (HCV).

There are a number of existing attempts to shield the needle tip after placement of the catheter. Such devices can be found in U.S. Pat. Nos. 7,658,725 and 7,736,342 assigned to Smiths Medical ASD, Inc. U.S. Pat. Nos. 9,095,683, 9,861,792 and publication No. US20180126125 assigned to C.R. Bard, Inc.

Enclosed needle devices that use an elongated needle guard housing to shield the needle tip by enclosing the needle cannula with a manually movable needle support have been well-accepted and are in widespread use, further improvement is desired to make the mechanism simple and easy to manufacture. Therefore, there remains a need for an improved needle catheter assembly that allows for insertion of the needle catheter assembly and renders the needle in a protected configuration while remaining simple and easy to use.

### BRIEF DESCRIPTION OF THE SEVERAL VIEWS OF THE DRAWINGS

The accompanying drawings, which are incorporated in and constitute a part of this specification, illustrate an exemplary embodiment of the invention and, together with a general description of the invention given above, and the detailed description given below, serve to explain the invention.

FIGS. 1A to 1B illustrate exemplary a catheter and needle respectively as basic components of a safety catheter-needle assembly.

FIGS. 2A to 2C illustrate a body member that couples to a needle hub.

FIGS. 3A to 3C illustrate a protective sheath for use with a variation of a catheter needle assembly as disclosed herein.

FIGS. 4A to 4D illustrate a catheter needle assembly transitioning from an unprotected/ready state to a protected or shielded state.

FIGS. 5A and 5B show an example of the operation of a variation of a safety assembly of the present disclosure in use.

FIGS. 6A to 6C illustrate an improved valve for use with a catheter.

FIGS. 7A to 7C illustrate exemplary basic components of a safety catheter-needle assembly as discussed herein.

FIGS. 8A and 8B illustrate an assembled safety catheter-needle assembly where the catheter is positioned over the dilator, which is positioned over the needle.

FIG. 8C shows a magnified view of the distal end of FIG. 8B.

FIGS. 9A and 9B illustrate an example of actuating the assembly to shield the sharp tip of the needle.

FIGS. 10A and 10B are drawings that illustrate a locking feature of the dilator relative to the needle.

### DETAILED DESCRIPTION OF THE INVENTION

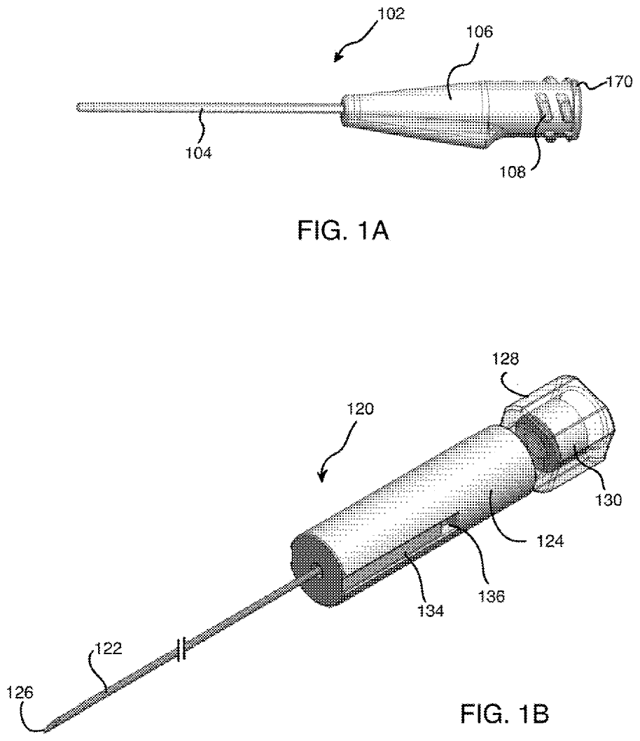

FIGS. 1A through 5B (which are not necessarily to scale in order to show the various components more readily), illustrate variations of needle assemblies that permit securing the needle in a protected configuration. The devices described herein provide for an improved catheter-needle-dilator assembly (or just a dilator-needle assembly) that permits insertion of a needle within a vein, determination of access to the vein via flashback and then allows rendering of the needle in a protected configuration without movement of the needle from the vein. The devices should be simple and easy to use. The locking dilator safety devices described herein also allow for deep access and ultrasound guidance. The locked dilator hoods over the needle bevel to reduce vein injury which provides a safety sheath for needlestick safety. The dilators described herein dilate the vein wall to “seal” the insertion site from infiltration.

FIGS. 1A to 1B illustrate exemplary basic components of a safety catheter-needle assembly as discussed herein. FIG. 1A illustrates a catheter 102 having a catheter tubing 104 or extrusion coupled to a catheter hub 106. The catheter hub 106 can include an internal luer taper as commonly known with a valve 170 seated within a chamber of the catheter hub 106. The catheter hub 106 can optionally include a threaded portion 108 on an exterior of the hub or extending from the hub (not shown).

FIG. 1B illustrates a needle 120 having a cannula 122 or needle shaft that ends in a sharp tip 126. The needle 120 includes a needle hub 124. In the illustrated variation, the needle hub 124 includes a flash chamber 128 with a porous plug (not shown in FIG. 1B). The plug can be positioned in a proximal end of the flash chamber 128, which is towards a proximal end of the needle hub 124. The plug 130 is similar to those known in the art and described in U.S. Pat. No. 7,736,342, incorporated by reference. In one variation, the plug 130 comprises a venting material that allows gas (such as air) to pass through, but retains blood or other fluids within the flash chamber 128. The plug 130 typically remains in place and prevents passage of fluids through the flash chamber 128 but alternate variations include configurations where the plug 130 can be removed, giving the medical caregiver access into and through the flash chamber 128 and needle cannula 24 as desired. In some variations, the flash chamber and/or needle hub can be entirely transparent or partially opaque to allow visualization of blood or fluid flow within the chamber and needle.

Alternatively, not shown, a guidewire can be positioned through the plug 130. In such a variation, the guidewire is slidable through the plug 130 while allowing the plug to prevent the passage of liquids in a proximal direction out of the flash chamber 128. As discussed below, the flash chamber 128 permits a medical caregiver to observe flow of blood into the chamber 128 once the sharp tip 126 of the needle 120 penetrates a blood vessel. In alternate variations, the flash chamber 128 can be detachable or separate from the needle hub 124. Alternatively, the flash chamber and/or needle hub can include one or more windows to allow visual confirmation of any substance within the needle/flash chamber.

FIG. 1B also illustrates the needle having one or more tracks/keyway 134 and a pocket 136. As described below, the track/keyway 134 allows for coupling of a body unit to the needle hub 1124.

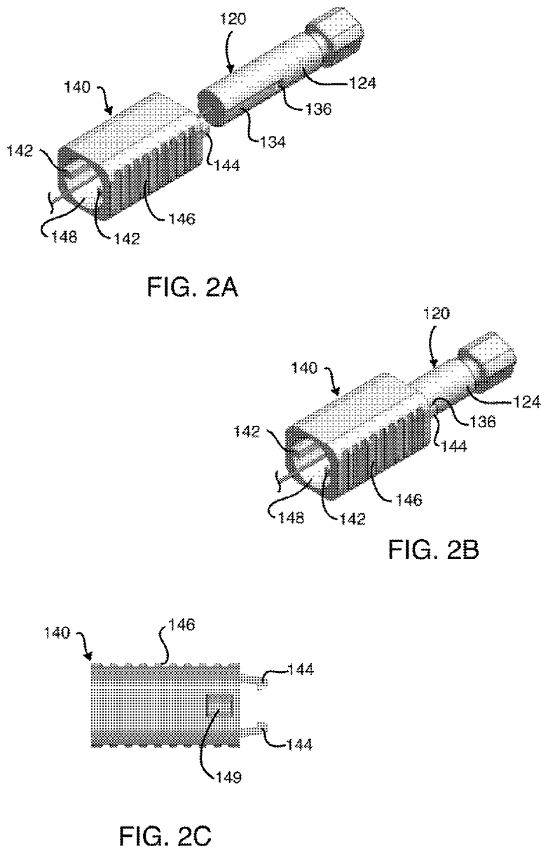

FIGS. 2A to 2C illustrate a body member 140 that couples to a needle hub 120. FIG. 2A illustrates an exploded assembly view of the body member 140 spaced from the needle hub 124 of a needle 120. The body member 140 can include a gripping surface 146, which, as described below, allows for manipulation of the needle-catheter assembly and movement of a protective dilator or sheath (not shown) over the distal end of the needle 120. The body member 140 includes a passage 148 that includes one or more rail structures 142. The rail structures slidably receive a dilator hub or sheath hub as described below. As shown, attachment features or clips 144 are advanced over the tracks 134 of the needle hub 124.

FIG. 2B illustrates the body member 140 positioned over the needle hub 124 such that the locking features 144 secure into pockets to prevent relative movement between the body member 140 and needle 120.

FIG. 2C illustrates a bottom view of the body member 140. In some variations, the locking features 144 extend from the rails 142. Alternatively, the locking features 144 and rails 142 can be separate structures. FIG. 2C also illustrates the body member as having a pocket 149. The pocket can receive a tab or other protrusion that is part of the dilator/sheath hub to lock the protective sheath/dilator in place relative to the needle 120.

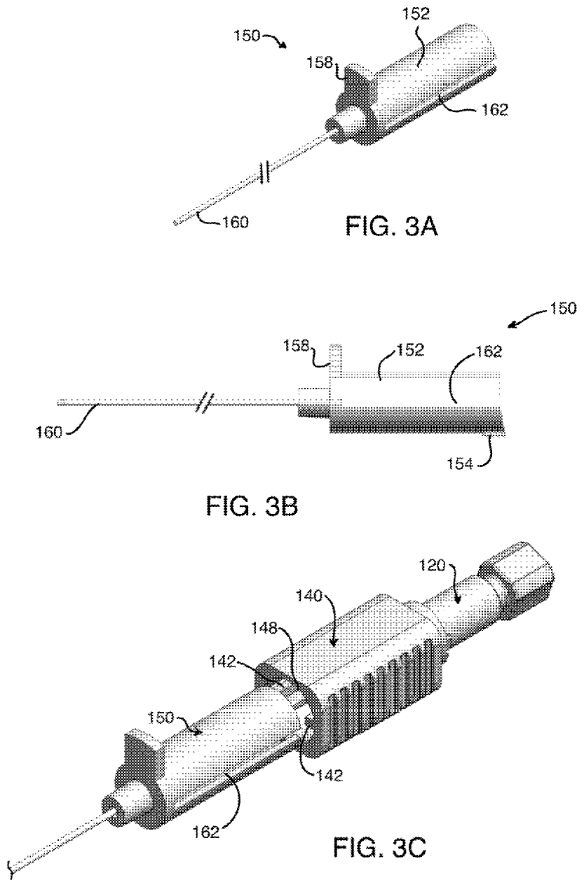

FIGS. 3A to 3C illustrate a protective sheath for use with a variation of a catheter needle assembly as disclosed herein. In this variation, the protective sheath 150 also functions as a dilator as the tip of the dilator tubing 160 expands tissue after entry of a needle as disclosed below. FIG. 3A illustrates an isometric view of a variation of a dilator 150 having a hub 152 with a protrusion 158 that can be used to move the dilator 150 relative to the catheter needle assembly. The hub 152 includes a plurality of grooves or slots 162. In the illustrated variations, there are two slots 162 that extend through a wall of the hub 152. However, alternate variations include recesses that do not extend through the hub 152. Alternatively, the slots 162 can be replaced with protrusions that define a slot or channel along the dilator hub 152.

FIG. 3B illustrates a side view of the dilator of FIG. 3B. As shown, the dilator body 152 can optionally include one or more locking tabs 154. As discussed below, the locking tab 154 is designed to prevent movement of the dilator/sheath relative to the needle once the dilator/sheath shields the sharp tip of the needle.

FIG. 3C illustrates the dilator 150 positioned within a passage 148 of the body member 140. As shown, the slots 162 align with the rails 142 within the body member 140. The body member 140 is coupled to a needle 120 as shown in FIG. 2B.

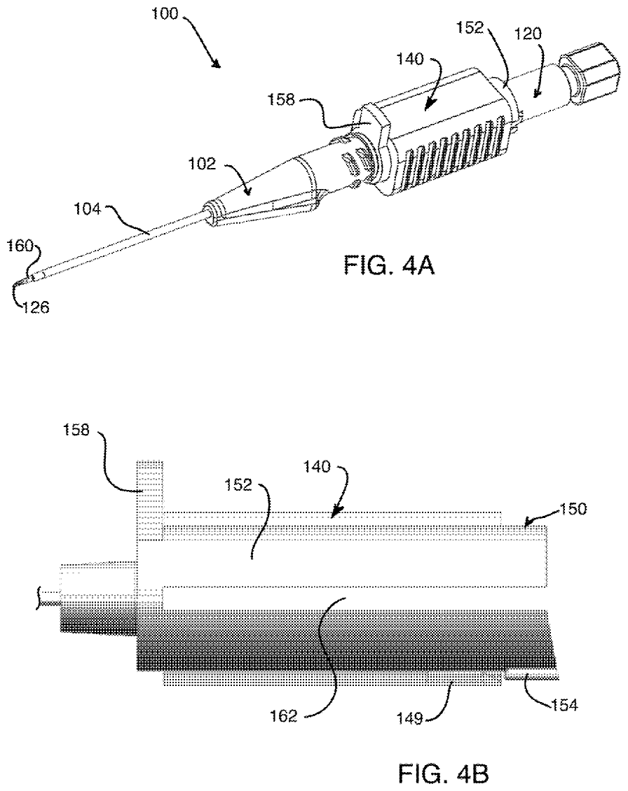

FIGS. 4A to 4D illustrate a catheter needle assembly 100 transitioning from an unprotected/ready state to a protected or shielded state. FIG. 4A illustrates a variation of a needle catheter assembly 100 as discussed herein where a catheter 102 is positioned on a dilator or sheath 160. (As noted above, the term sheath is intended to include a dilator for purposes of the variations discussed herein.) The catheter 102 and sheath 160 are positioned on a needle 120 that terminates at a distal sharp tip 126. In this variation, the catheter tubing 104 extends proximal to the dilator or sheath tubing 160. The protrusion 158 of the dilator hub 152 is positioned between the catheter 102 (or valve) and the body member 140. As discussed herein, the body member 140 is coupled to the needle 120 so that movement or manipulation of the body member 140 allows for insertion of the catheter assembly 100 to penetrate a vessel (as discussed below). The configuration shown in FIG. 4A is considered the unprotected/ready state since the needle tip 126 is protruding from the dilator 160 and catheter tubing 104 such that the assembly 100 is ready to penetrate tissue.

FIG. 4B illustrates a cross-sectional view of the body member 140 and dilator hub 152 positioned therein. As shown, the dilator hub 154 includes a tab 154. In this variation, the tab 154 is positioned outside of the body member 140. However, alternate variations are within the scope of this disclosure. The construction of the catheter assembly 100 permits alignment of the tab 154 with the pocket 149 of the body member 140.

FIG. 4C illustrates the catheter assembly 100 of FIG. 4A where the protrusion 158 of the dilator hub 152 is moved relative to the body member 140. The protrusion 158 can either be advanced distally to the body member 140 or the body member 410 can be moved proximally. In any case, because the needle 120 is coupled to the body member 140, the relative movement between the protrusion 158/dilator hub 152 and the body member 140 causes positioning of the needle tip (not shown) within the dilator tubing 160. This action places the assembly 100 in a protected or shielded state. Variations of the assembly 100 allow for the catheter 102 to be positioned on the assembly 100 during shielding. Alternatively, the catheter can be removed from the assembly 100 during the shielding process.

FIG. 4D illustrates the cross-sectional view of FIG. 4B where the dilator hub 152 is moved relative to the body member 140. As shown, the tab 154 of the dilator hub 154 is moved within the pocket 149 of the body member 140 to effectively lock the dilator 150 relative to the body member 140. Because the body member 140 is coupled to the needle (not shown), the dilator 150 becomes locked relative to the needle as well.

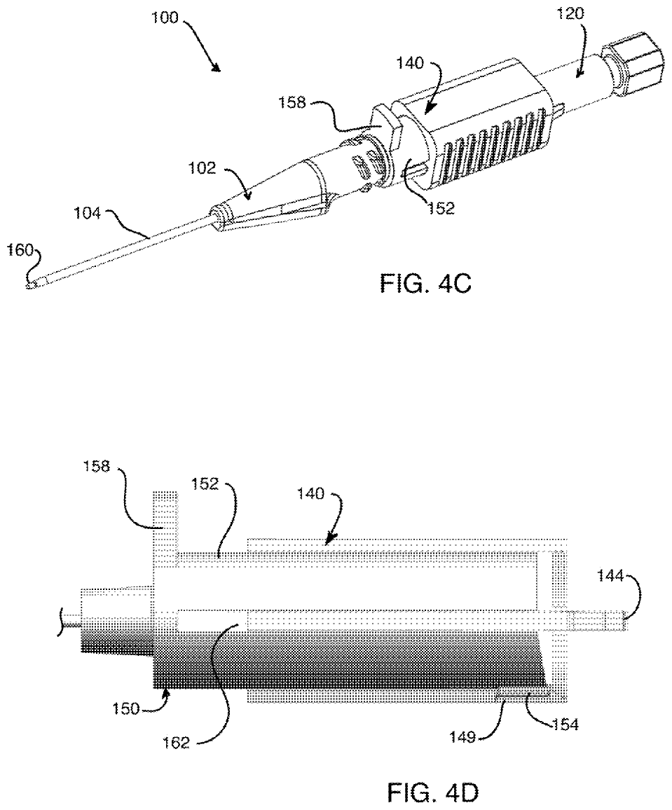

FIGS. 5A and 5B show an example of the operation of a variation of a safety assembly 100 of the present disclosure in use. FIG. 5A illustrates a situation where a medical caregiver administers the assembly 100 through tissue 20 of a patient and directly into a vessel 22. The assembly 100 is in a ready or unprotected state. The caregiver will confirm placement of the sharp tip 126 of the needle cannula within the vessel 22 by observing for blood flashback within the needle hub. Flashback occurs when blood within the vessel 22 enters a lumen of the needle and flows into the flashback chamber. Alternatively, the needle cannula can have one or more separate holes in a wall of the cannula to provide for entry of blood into the needle lumen and into the flash chamber. Once the caregiver observes flashback (or flow of other liquids into the chamber), the caregiver can maintain the needle assembly 100 in position and advance the actuation tab of the dilator as discussed above. FIG. 5B illustrates the state of the device assembly 100 when flashback was observed and the actuation tab (not shown—158 in FIG. 4A) produces relative movement between the dilator assembly including the dilator tubing 160 and the sharp tip 126 of the needle cannula. In one variation, the actuation tab is driven forward to shield the needle. Advancing the dilator tubing 160 ensures that the distal end of the device assembly 100 is maintained within the vessel 22. In alternate variations, the advancement tab can be held stationary and the needle hub can be moved to cause the sharp distal tip 126 to move proximal to the opening of the dilator tubing 160.

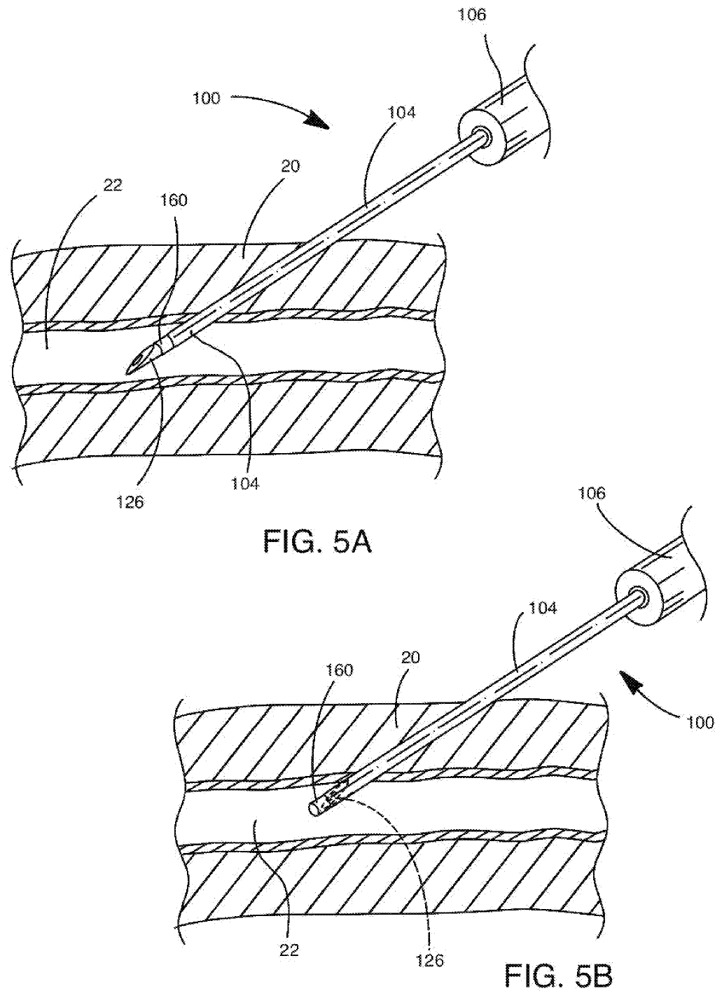

FIGS. 6A-6C illustrate an improved valve for use with any catheters described herein. Typically, such a catheter 102 is used with a male luer. The catheter 102 includes a catheter hub 106 having a chamber 112 with a proximal surface 110 defining an open proximal end. The chamber 112 is in fluid communication with a catheter tubing 104 that is coupled the catheter. The tubing includes one or more lumens in fluid communication with the chamber 112. The valve or septum valve 170 includes a barrier layer (or septum) 172 at a distal end. The barrier layer 172 can have one or more slits 174. The illustrated variation shows a barrier layer 174 with 3 slits that form three leaflet structures or flaps. However, variations of the valve 170 include any number of slits forming any number of leaflets. The barrier layer 172 generally includes a flexible or semi-flexible material that is compatible with exposure to blood, medicaments, and other fluids commonly encountered during catheterization/infusion procedures.

As shown in FIG. 6B, the valve includes a wall portion 176 extending proximally from the barrier layer 172 and defines a valve cavity 182. A flange portion 180 is formed around the wall 176 at a proximal end of the valve 170. The flange portion comprises a diameter greater than a diameter of the wall portion. Variations of the valve 170 include a flange portion 180 that encircles the valve 170. Alternatively, the flange portion 180 can include openings or segments such that it is not circumferentially continuous about the wall.

FIG. 6C shows the valve 170 coupled to the catheter hub 106 such that the flange portion 180 engages the proximal surface 110 of the catheter hub 106 and is exterior to the chamber 112 of the hub 106. The wall portion 176 of the valve 170 engages a surface of the chamber. The valve 170 can be affixed to the catheter at various points. For example, variations of the assembly include a valve 170 that is only affixed to the catheter hub 106 at the flange portion 180 using an adhesive or joining material where the wall portion 196 is simply positioned against a wall of the chamber 140. Alternatively, or in combination, the valve 190 can be affixed to the catheter hub 106 at the exterior wall portion 176. In an alternate variation, the valve 170 can simply be press-fit into the catheter hub 106. Any number of features known to those in the art can be used to facilitate seating of the valve 170 within the catheter hub 106 (e.g., pockets, ribs, increased frictional resistance of the surface of the valve or chamber, etc.)

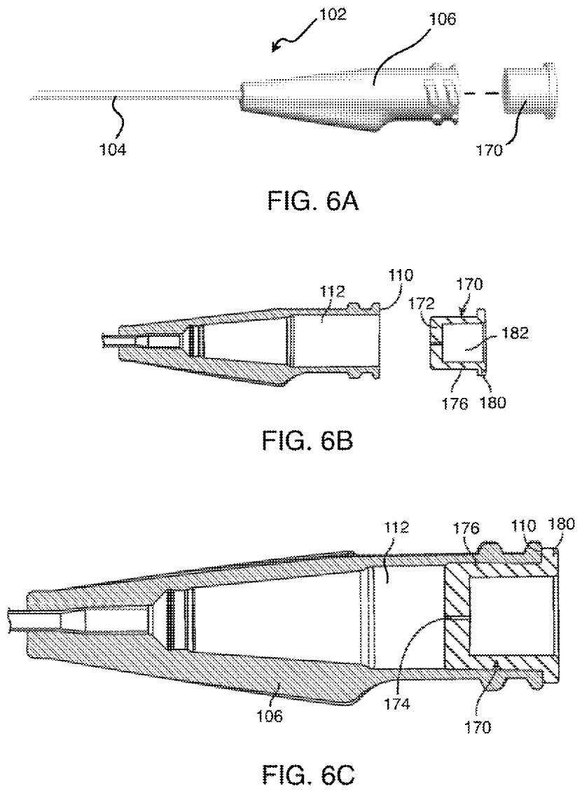

FIGS. 7A to 7C illustrate another example of a safety catheter-needle assembly as discussed herein. FIG. 7A illustrates a catheter 102 having a catheter tubing 104 or extrusion coupled to a catheter hub 106.

FIG. 7B illustrates a needle 120 having a cannula 122 or needle shaft that ends in a sharp tip 126. The needle 120 includes a needle hub 124. In the illustrated variation, the needle hub 124 includes a flash chamber 128 with a porous plug 130. The plug 130 is positioned in a proximal end of the flash chamber 128, which is towards a proximal end of the needle hub 124. The plug 130 is similar to those known in the art and described in U.S. Pat. No. 7,736,342, incorporated by reference). The plug 130 comprises a venting material that can pass gas (such as air) but retains blood or other fluids within the flash chamber 128. The plug 130 typically remains in place against passage of fluids through the flash chamber 128 but alternate variations include configurations where the plug 130 can be removed, giving the medical caregiver access into and through the flash chamber 128 and needle cannula 24 as desired. Alternatively, not shown, a guidewire can be positioned through the plug 130. In such a variation, the guidewire is slidable through the plug 130 while allowing the plug to prevent the passage of liquids in a proximal direction out of the flash chamber 128. As discussed below, the flash chamber 128 permits a medical caregiver to observe flow of blood into the chamber 128 once the sharp tip 126 of the needle 120 penetrates a blood vessel. In alternate variations, the flash chamber 128 can be detachable or separate from the needle hub 124.

FIG. 7C shows a dilator 150 having a dilator hub 152 at the end of a dilator tubing 160. In the illustrated figure, the dilator hub 152 is shown in two pieces for the purposes of illustration only. The dilator hub 152 further includes a locking feature, which in this illustrated variation comprises a set of locking tabs 154, 156. However, the locking feature can comprise a single locking structure or tab or any similar structure that allow locking of the dilator 150 relative to the needle 120 as described herein. The dilator hub 152 further includes a tab or protrusion 152 that allows a medical caregiver to move the dilator 150 relative to the needle 120.

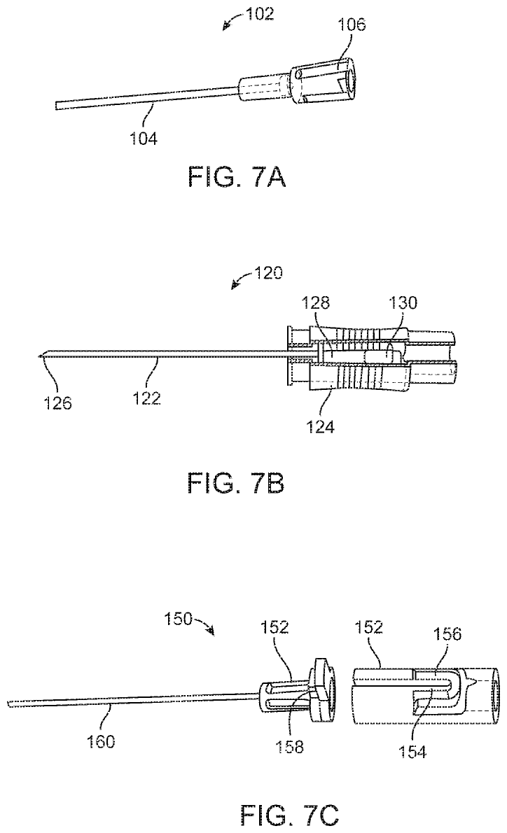

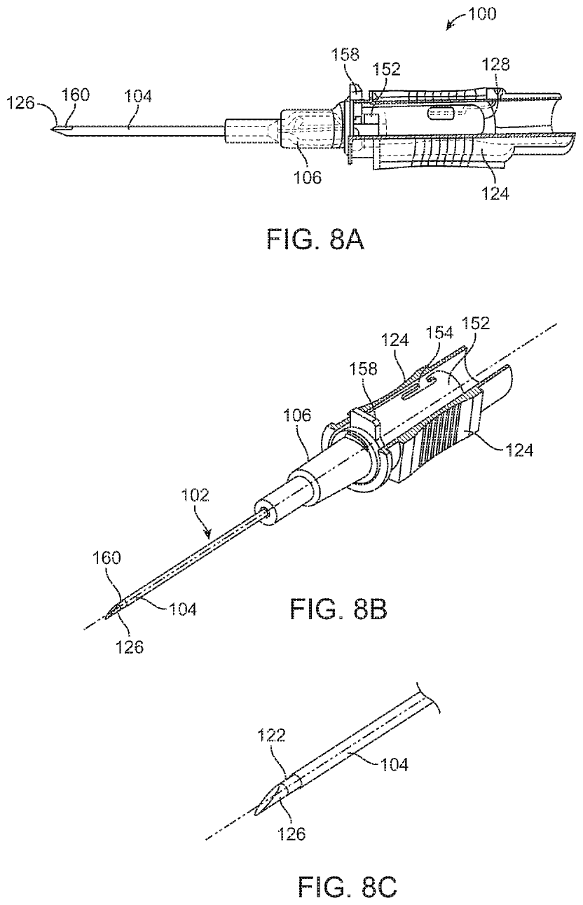

FIGS. 8A and 8B illustrate an assembled safety catheter-needle assembly 100 where the catheter is positioned over the dilator, which is positioned over the needle. FIG. 8A illustrates a top view of the assembly 100, while FIG. 8B provides an illustration of the assembly 100 in an isometric view. In this arrangement, the dilator hub 152 is positioned over the flash chamber 128 in a sliding arrangement as discussed below. Therefore, a medical caregiver can grasp the needle hub 124 and, with a single hand, advance the actuation tab 158 to produce relative movement between the needle and dilator to position the needle in a protected state as discussed below. The locking feature 154 shown in FIG. 8B is shown rotated for illustrative purposes. As discussed below, the locking feature 154 engages a portion of the needle hub 124 that allows the medical caregiver to lock the dilator in a safety position such that the sharp tip is covered.

In the variation shown in FIGS. 8A and 8B, the catheter hub 106 is positioned over the dilator hub such that the device is in a “ready state” where the sharp tip is exposed at a distal end of the dilator tubing 160, which is adjacent to the catheter tubing 104. FIG. 8C shows a magnified view of the distal end of FIG. 8B to further illustrate the sharp tip 126 of the needle cannula extending beyond the dilator tubing 122 with the catheter tubing 104 adjacent to the end of the dilator tubing 122.

While not shown, the assembly 100 can include any number of protective guards over the sharp tip 126. For example, the assembly 100 can be provided in the ready configuration with a disposable guard positioned over the sharp tip 126, and optionally over the dilator 160 and catheter tubing 104. Therefore, when the medical caregiver is ready to insert the assembly 100, the caregiver can remove and discard the disposable guard and insert the device.

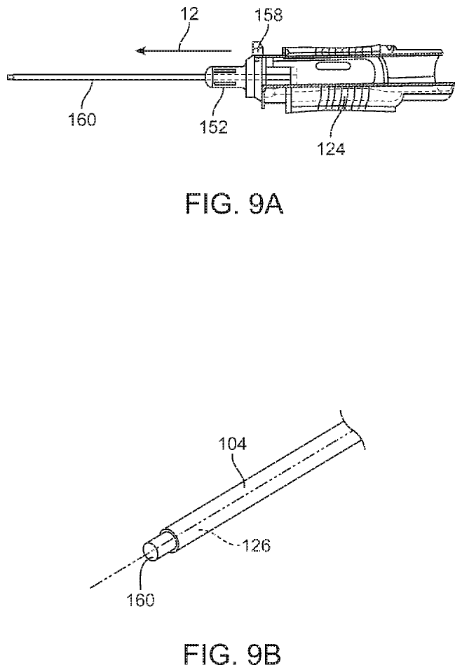

FIGS. 9A and 9B illustrate an example of actuating the assembly to shield the sharp tip of the needle. FIG. 9A illustrates the needle and dilator (the catheter is omitted for purposes of illustration). As shown, movement 12 of the actuator tab 158 relative to the needle hub 124 causes movement of the dilator hub 152 and dilator tubing 160 relative to the needle such that the needle is shielded by the dilator tubing 160. FIG. 9B illustrates a state of the device when in the protected configuration with the sharp tip 126 within the catheter tubing 104 and dilator tubing 160.

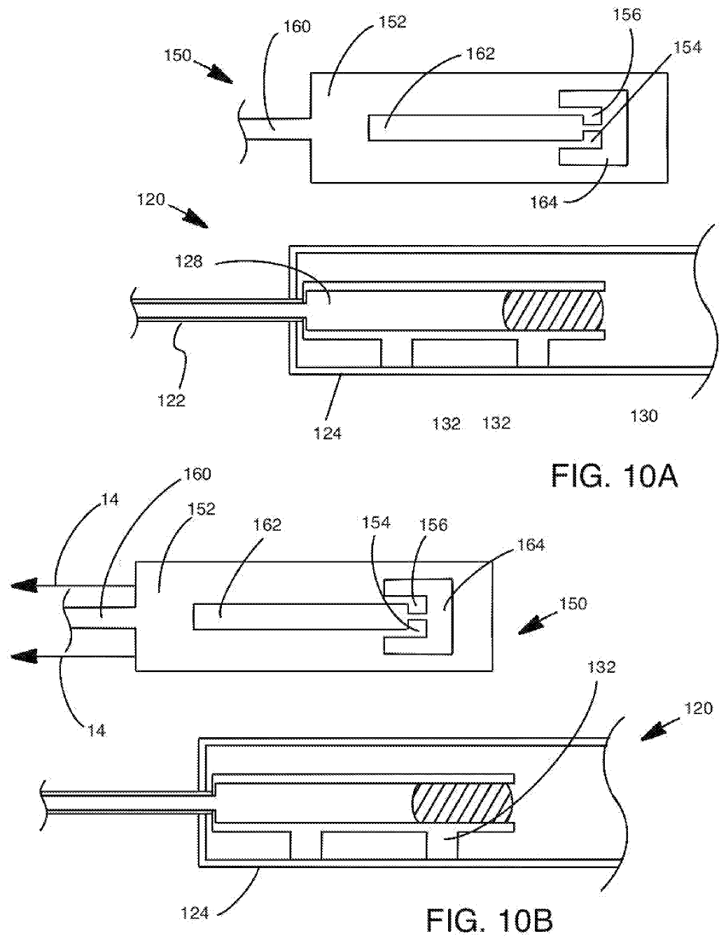

FIGS. 10A and 10B are drawings that illustrate a locking feature of the dilator 150 relative to the needle 120. The catheter is not illustrated for purposes of clarity, but typically the catheter is positioned on the dilator 150 as described above. Also, the dilator 150 is shown outside of the needle hub 124 and rotated relative to the needle hub 124 to better illustrate the locking action. As noted below, during assembly the dilator 150 is rotated 90 degrees about an axis of the dilator tubing 160 so that an alignment slot 162 of the dilator slides about flash chamber support members 132. In the ready or initial configuration, the locking features 154 and 156 of the dilator hub 152 are located proximally to the flash chamber 128 and supports 132. Upon placing the sharp tip in a vessel, blood will enter the chamber 128, which confirms proper placement of the needle cannula within the vessel. At this point, the caregiver maintains the needle assembly in place and uses the actuator tab (not shown) to secure the sharp tip 126 of the needle cannula 122 within the dilator tubing 160 (see FIG. 4B).

As shown in FIG. 10B, this action causes distal movement 14 of the dilator hub 152 (or relative movement between the dilator hub 152 and needle hub 124). This movement also causes locking tabs 154 and 156 to deflect away from each other when advanced against the rearmost flash chamber support 132. Continued advancement 14 of the dilator hub 152 (or actuation tab) moves the locking feature (tabs 154 156) distal to the rearmost flash chamber support 132 such that the support 132 is now positioned within the clearance opening 164 adjacent to the locking features (154, 156) and the locking features are no longer deflected by the support such that they return to their initial position and provide a “locking” of the dilator since the features are now on the opposite side of the support. This effectively locks the dilator hub 152 and dilator tubing 160 distal to the sharp tip of the needle, as discussed above, while the needle remains within the vessel 22. Once locked, the needle is shielded within the dilator and the two components are removed and disposed with the catheter remaining in the vessel.

In an additional variation, the catheter hub can engage the dilator, such that advancement of the catheter hub from the assembly pulls the dilator relative to the needle assembly, causing the dilator to enter into the locked configuration and shield the distal needle tip. The catheter would then separate or disengage from the locked dilator and move into the vein or artery to the desired length. The locked dilator/needle assemblies would then be withdrawn from the catheter. Alternatively, this configuration can also allow the act of withdrawing the needle and dilator to cause the dilator to move relative to the needle such that the needle and dilator are positioned in the protective state that shields the needle tip.

We claim:

1. A catheter assembly compromising:
a dilator comprising a dilator tubing extending from a dilator hub, the dilator hub comprising a locking feature and an actuator tab;
a needle comprising a needle cannula having a sharp tip and a needle hub located at a proximal end of the needle cannula, the needle cannula having a needle lumen extending therethrough and in fluid communication with a flash chamber in the needle hub, wherein in a ready position the needle is positioned within the dilator and the sharp tip extends distally to the dilator tubing; and
a catheter comprising a catheter tubing extending from a catheter hub, the catheter positioned over the dilator; and
wherein displacement of the dilator hub relative to the needle hub until the locking feature to engages a portion of the needle hub which positions the dilator in a locked configuration where a distal end of the dilator tubing extends beyond a sharpened end.

2. The catheter assembly of claim 1, where the needle hub further comprises a body member coupled to the needle hub, wherein the body member is positioned adjacent to the dilator hub such that distal movement of the body member moves the dilator hub distally, wherein relative movement of the dilator hub to the body member seats the locking feature within a recess in the body member causing locking of the body member with the dilator hub.

3. The catheter assembly of claim 2, wherein the dilator hub is located exteriorly to the needle hub and within a passage of the body member such that prior to locking, the dilator hub is slidable within the passage of the body member.

4. The catheter assembly of claim 3, wherein the passage of the body member comprises at least one rail member and where the dilator hub comprises at least one slot, wherein the at least one slot receives the at least one rail member such that the dilator hub is prevented from rotating relative to the body member.

5. The catheter assembly of claim 4, wherein a portion of the at least one rail member is coupled to the needle hub through the at least one slot.

6. The catheter assembly of claim 2, wherein the actuator tab extends radially beyond the body member, wherein the actuator tab permits distal movement of the dilator hub relative to the body member until locking of the body member with the dilator hub.

7. The catheter assembly of claim 1, wherein the locking feature engages a portion of the flash chamber to assume the locked configuration.

8. The catheter assembly of claim 1, wherein the needle hub is configured to slide about the flash chamber.

9. The catheter assembly of claim 1, further comprising a plug located in the flash chamber, where the plug permits passage of gasses therethrough while preventing the passage of liquids therethrough.

10. A method of accessing a blood vessel, the method comprising:
providing a catheter assembly comprising a needle having a needle cannula having a sharp tip and a needle hub located at a proximal end of the needle cannula, the needle cannula having a needle lumen extending therethrough and in fluid communication with a flash chamber in the needle hub, a dilator comprising the catheter assembly further including a dilator tubing extending from a dilator hub, the dilator hub comprising a locking feature and an actuator tab, where the dilator tubing is positioned proximally to the sharp tip of the needle cannula, the catheter assembly further including a catheter having a catheter tubing extending from a catheter hub, the catheter being positioned exterior to the dilator, wherein in a ready position the sharp tip extends distally to the dilator tubing, the catheter assembly further comprising a catheter having a catheter tubing extending from a catheter hub, the catheter being positioned over the dilator;
advancing a catheter assembly into tissue such that the sharp tip of the needle cannula enters a blood vessel;
observing the flash chamber for blood from the blood vessel;
upon observing the presence of blood in the flash chamber, and without moving the sharp tip of the needle cannula from the blood vessel, advancing the dilator distally to the needle such that the dilator tubing extends distally beyond the sharp tip to a locked position; and
advancing the dilator and needle cannula in the locked position as needed to allow a distal tip of the catheter to enter the blood vessel;
stabilizing the needle cannula and dilator in the locked position then advancing the catheter distally into the blood vessel to a desired length; and
withdrawing the needle and dilator from the blood vessel and tissue such that the catheter provides an access path into the blood vessel.

11. The method of claim 10, where the needle hub further comprises a body member coupled to the needle hub, wherein advancing the dilator distally to the needle to the locked position comprises advancing the actuator tab relative to the body member such that the locking feature of the dilator hub engages a recess in the body member to cause the locked position.

12. The catheter assembly of claim 11, wherein the dilator hub is located exteriorly to the needle hub and within a passage of the body member such that prior to the locked position, the dilator hub is slidable within the passage of the body member.
